(19) United States
(12) Patent Application Publication
(10) Pub. No.: US 2005/0277137 A1
(43) Pub. Date: Dec. 15, 2005

(54) Diagnostic multimarker serological profiling

(75) Inventors: Anna Lokshin, Pittsburgh, PA (US); Elieser Gorelik, Pittsburgh, PA (US)

Correspondence Address: THE WEBB LAW FIRM, P.C., 700 KOPPERS BUILDING, 436 SEVENTH AVENUE, PITTSBURGH, PA, 15219, US

(73) Assignee: University of Pittsburgh

(21) Appl. No.: 11/104,874

(22) Filed: Apr. 13, 2005

Related U.S. Application Data
(63) Continuation-in-part of application No. 10/918,727, filed on Aug. 13, 2004.
(60) Provisional application No. 60/495,547, filed on Aug. 15, 2003.

(51) Int. Cl. C12Q 1/68; G01N 33/574

(52) U.S. Cl. 435/6; 435/7.23

(57) ABSTRACT

The present invention provides a novel multianalyte LabMAP™ profiling technology that allows simultaneous measurement of multiple markers. In particular, a method is provided for diagnosing the presence of pancreatic cancer in a patient by measuring serum levels of markers in a blood marker panel comprising at least IP-10, HGF, IL-8, βFGF, IL-12p40, TNFRI, TNFRII, Eotaxin, MCP-1 and CA 19-9, wherein a significant increase in the serum concentrations of IP-10, HGF, IL-8, βFGF, IL-12p40, TNFRI, TNFRII, and CA 19-9 compared to healthy matched controls, and a significant decrease in the serum levels of Eotaxin and MCP-1 compared to healthy matched controls, indicates a probable diagnosis of pancreatic cancer in the patient. Also provided is a method to distinguish pancreatic cancer from chronic pancreatitis by measuring serum levels of markers in a blood marker panel. The present invention further provides a method of predicting the onset of clinical pancreatic cancer in a patient by determining the change in concentration at two or more time points of serum levels of markers on a blood marker panel.

### CROSS REFERENCE TO RELATED APPLICATIONS

This is a Continuation-In-Part Patent Application of U.S. Pat. Ser. No. 10/918,727 filed Aug. 13, 2004, which claims the benefit of U.S. Provisional Patent Application No. 60/495,547, filed Aug. 15, 2003, which is incorporated herein by reference in its entirety.

### BACKGROUND OF THE INVENTION

1. Field of the Invention

The present invention relates to methods and reagents for a multianalyte assay for the rapid, early detection of cancer.

2. Description of Related Art

Pancreatic adenocarcinoma (PA) is the fifth leading cause of cancer death in the United States, accounting for more than 26,000 deaths a year. The prognosis for patients with PA is poor, with reported one-year survival rates between 5% and 10% and an overall five-year survival rate of 3% for all stages, one of the poorest five-year survival rates of any cancer. At the time of diagnosis, over four-fifths of patients with PA have clinically apparent metastatic disease. Among patients whose disease is considered to be resectable, 80% will die of recurrent tumor within 2 years. Factors which appear to be improving long-term survival include improved pancreatectomy technique, earlier detection, reduced perioperative mortality and decreased blood transfusions.

The main risk factor for PA is smoking, i.e., about 30% of PA is thought to be a direct result of cigarette smoking. Other risk factors include: age, i.e., most often seen in people older than 60; gender, i.e., men are 30% more likely to develop pancreatic cancer; chronic pancreatitis; diet, i.e., a diet high in meats and fats appears to increase risk; diabetes mellitus; exposure to some industrial chemicals, such as certain pesticides and petroleum products; and family history, i.e., an inherited tendency may be a factor in 5% to 10% of cases.

Early diagnosis of PA is difficult but essential in order to develop improved treatments and a possible cure for this disease. Currently, the ability to detect early lesions for resection remains a diagnostic challenge despite the advances in diagnostic imaging methods like ultrasonography (US), endoscopic ultrasonography (EUS), dualphase spiral computer tomography (CT), magnetic resonance imaging (MRT), endoscopic retrograde cholangiopancreatography (ERCP) and transcutaneous or EUS-guided fine-needle aspiration (FNA). Furthermore, distinguishing PA from benign pancreatic diseases, especially chronic pancreatitis, is difficult because of the similarities in radiological and imaging features and the lack of specific clinical symptoms for PA.

Early detection and treatment has lead to improved overall survival for breast, colon, lung, and prostate cancers (Etzioni, R. et al., Nat. Rev. Cancer 3:243-252, 2003). There is retrospective data to support the efficacy of early detection and treatment in patients with pancreatic cancer as well. In one of the largest retrospective studies of prognostic factors, performed on 616 patients with pancreatic cancer undergoing potentially curative resection, Sohn et al. showed that survival was markedly improved in early stage patients who had small tumors, negative resection margins and no lymph node involvement (31% vs 15% five-year survival) (Sohn, T. A. et al., J Gastrointest. Surg. 4:567-579, 2000). Ariyama et al. have reported 100% five-year survival in patients undergoing resection of pancreatic tumors less than 1.0 cm (Ariyama, J. et al., Pancreas 16: 396-401 1998). Early experience with screening populations at very high risk of pancreatic cancer with invasive techniques like endoscopic ultrasound and endoscopic retrograde cholangiopancreatography have been encouraging (Rulyak, S. J. et al., Gastrointest. Endosc. 57: 23-9 2003). The general requirements for performance of a screening test for pancreatic cancer have been examined by Lowenfels (Lowenfels A. B. et al., J. Natl. Cancer Inst., 89:442-6, 1997). In his analysis he assumed screening for pancreatic cancer starting at the age of 50, a population with 10% lifetime risk of developing the disease, and a 40-50% survival rate after curative surgery. He concluded that a screening test with a sensitivity and specificity >90% range could result in an additional 0.69 years of life.

A variety of serum tumor markers that correlate with the presence of pancreatic cancer have been described in the literature. Probably the most widely used is CA 19-9. Most studies, using a variety of cut-off points, have found a high degree of correlation between elevated CA 19-9 levels and the presence of pancreatic cancer. Although sensitivity and specificity for CA 19-9 have been reported to be between 70-90% and 90%, respectively (Kim, H. J. et al., Am. J. Gastroenterol. 94: 1941-6 1999), there is a high degree of overlap between CA 19-9 serum levels in pancreatic cancer and a variety of benign inflammatory conditions of the pancreas, notably chronic pancreatitis, and thus the clinical applicability of CA 19-9 as a specific screening marker for pancreatic cancer is quite limited. Multiple other single serum markers, such as TPA, TIMP-1, CEA, CA-125, mesothelin, osteopontin and MIC-1, also have been examined. However, none of these serum markers has been found to be of sufficient sensitivity and specificity to warrant clinical use at the present time.

Chronic pancreatitis with pancreatic inflammation is the most prominent clinical confounding condition that needs to be distinguished when making the diagnosis of pancreatic cancer. The failure of single serum markers to accurately distinguish between the complex biology of pancreatic cancer and chronic pancreatitis has lead investigators to examine the performance of combinations of markers. The performance of CA 19-9 in combination with CEA and CA 72-4 has been reported (Hayakawa, T. et al., Int. J. Pancreatol., 25: 23-9, 1999). This combinatorial assessment of relevant markers improved both sensitivity and specificity of the detection of pancreatic cancer. The maximal detection power achieved in the above study was 89% sensitivity/87% specificity, well below the required threshold for screening populations at medium and average risk of pancreatic cancer.

A causative or associative role for chronic inflammation and the development/progression of many adult neoplasms including pancreatic cancer has been postulated (Farrow, B. et al., Surg. Oncol., 10: 153-69, 2002; McMillan, D.C. et al., Nutr. Cancer 41: 64-9, (2001). A recent large population-based study demonstrated a definitive association between elevated serum levels of the inflammatory marker C-reactive protein and the development of colon cancer (Erlinger, T. P. et al., JAMA, 291: 585-90, 2004). This study suggests that markers of inflammation may be used as early signs of neoplasia. Furthermore, significant alterations in the levels of individual serum cytokines have been reported in pancreatic cancer (R. T. Penson, R. T. et al., Int. J. Gynecol. Cancer, 10: 33-41, 2000). There exists a critical need, therefore, to provide a relatively non-invasive screening test having high sensitivity and specificity in order to facilitate early diagnosis of pancreatic cancer.

Based on previous studies by the inventors demonstrating that combining CA 125 with a panel of cytokines resulted in improved sensitivity and specificity in early diagnosis of ovarian cancer (Gorelik, E. et al., Cancer Epidemiology Biomarkers and Prevention, In Press 2004), the inventors hypothesized that a panel comprised of cytokines, chemokines, and angiogenic factors could serve as cancer biomarkers to distinguish patients with pancreatic cancer from chronic pancreatitis and healthy controls.

### SUMMARY OF THE INVENTION

The present invention fulfills this need by providing methods for analyzing multiple serum markers using a novel LabMAP™ technology (Luminex Corp., Austin, Tex.) in order to provide a diagnostic assay for pancreatic cancer. The multiplexed cytokine panels offer a high predictive power for discrimination of pancreatic cancer from both healthy controls and from chronic pancreatitis. The methods of the present invention allow for rapid, early diagnosis of pancreatic cancer that have sufficient sensitivity and specificity to be clinically useful in disease diagnosis. The novel multianalyte LabMAP™ profiling technology allows for simultaneous measurement of multiple biomarkers in serum. The methods involve analysis of panels of markers including cytokines, chemokines, growth and angiogenic factors in combination with CA 19-9, in sera of pancreatic cancer patients, patients with chronic pancreatitis, and matched control healthy patients, in which the simultaneous measurement of panels of inflammatory and angiogenic factors is able to distinguish pancreatic cancer from healthy controls with a high sensitivity of 85.7% and specificity of 92.3%, which is superior to CA 19-9 alone. Furthermore, the multianalyte panels allow for the discrimination of pancreatic cancer from chronic pancreatitis with a high sensitivity of 98% and specificity of 96.4%.

In particular, a method of diagnosing the presence of pancreatic cancer in a patient is provided, comprised of measuring serum levels of markers in a blood marker panel comprising two or more, three or more, four or more, five or more, six or more, seven or more, eight or more, nine or more of IP-10, HGF, IL-8, bFGF, IL-12p40, TNFRI, TNFRII, Eotaxin, MCP-1 and CA 19-9, wherein a significant increase in the serum concentrations of IP-10, HGF, IL-8, βFGF, IL-12p40, TNFRI, TNFRII, and CA 19-9 in the patient compared to healthy matched controls, and a significant decrease in the serum levels of Eotaxin and MCP-1 in the patient compared to healthy matched controls, indicates a probable diagnosis of pancreatic cancer in the patient.

Also provided is a method to distinguish pancreatic cancer from chronic pancreatitis, comprised of measuring serum levels of markers in a blood marker panel from a patient comprising two or more, three or more, four or more, five or more, six or more, seven or more, or eight or more of IL-6, IL-8, IFNγ, TNFα, Eotaxin, MCP-1, MIP-1α, MIP-1β, and EGF, wherein a significant decrease in the serum levels of IL-6, IL-8, IFNγ, TNFα, Eotaxin, MCP-1, MIP-1α, MIP-1β, and EGF in the patient compared to patients with chronic pancreatitis, a significant increase in the serum levels of IP-10 in the patient compared to patients with chronic pancreatitis, and no significant difference in the serum levels of CA 19-9 in the patient compared to patients with chronic pancreatitis, indicates a probable diagnosis of pancreatic cancer in the patient.

Also provided is a method of predicting the onset of clinical pancreatic cancer in a patient, comprised of determining the change in concentration at two or more time points of two or more, three or more, four or more, five or more, six or more, seven or more, eight or more, or nine or more of IP-10, HGF, IL-8, b FGF, IL-12p40, TNFRI, TNFRII, Eotaxin, MCP-1 and CA 19-9 in the patient's blood between the two time points, wherein an increase in the concentration of IP-10, HGF, IL-8, bFGF, IL-12p40, TNFRI, TNFRII, and CA 19-9, and a decrease in the concentration of Eotaxin and MCP-1 in the patient's blood between the two time points are predictive of the onset of pancreatic cancer.

Also provided is a method for comparing the serum levels of the markers set forth herein in a blood marker panel with levels of the same markers in one or more control samples by applying a statistical method such as linear regression analysis, classification tree analysis and heuristic naïve Bayes analysis.

Also provided is an array comprised of binding reagent types specific to any two or more, three or more, four or more, five or more, six or more, seven or more, eight or more, nine or more, ten or more, eleven or more, twelve or more, thirteen or more, fourteen or more, or fifteen or more of IP-10, HGF, IL-6, IL-8, bFGF, IL-12p40, IFNγ, TNFα, TNFRI, TNFRII, Eotaxin, MCP-1, MIP-1α, MIP-1β, EGF and CA 19-9, wherein each binding reagent type is attached independently to one or more discrete locations on one or more surfaces of one or more substrates. The substrates may be beads comprising an identifiable marker, wherein each binding reagent type is attached to a bead comprising a different identifiable marker than beads to which a different binding reagent is attached. The identifiable marker may comprise a fluorescent compound or a quantum dot.

### BRIEF DESCRIPTION OF THE DRAWINGS

Table 1 provides summary statistics for serum cytokines by disease states;

Table 2 provides predictive values for individual serum markers for pancreatic cancer;

FIG. 1 shows serum levels of cytokines and growth factors in healthy controls, pancreatic cancer patients and patients with chronic pancreatitis. Sera were collected from 54 patients with pancreatic cancer, 22 patients with chronic pancreatitis and from 26 age, sex and smoking status-matched healthy controls. Circulating concentrations of cytokines and growth factors were measured using LabMAP™ technology. Measurements were performed twice. Horizontal lines indicate mean values. PanCA—pancreatic cancer; CP—chronic pancreatitis denotes statistical significance between controls and pancreatic cancer patients (when positioned over PanCa) or between patients with pancreatic cancer and patients with chronic pancreatitis (when positioned over CP), * P<0.05; ** P<0.01; *** P<0.001; and

FIG. 2 shows ROC curves discriminating pancreatic cancer from healthy controls (A) and chronic pancreatitis (B). ROC curves are presented for biomarker panels (multiplex) and for CA 19-9 alone. Presented are results from 10-fold cross validation of classification tree analysis of pancreatic cancer versus healthy controls (FIG. 2A) and chronic pancreatitis (FIG. 2B).

### DETAILED DESCRIPTION OF THE INVENTION

The present invention provides for the first time a multifactorial assay for early and rapid diagnosis of pancreatic cancer with sufficient sensitivity and specificity to be clinically useful in disease diagnosis.

The method of the present invention employs a novel multianalyte Luminex LabMAP™ profiling technology (Luminex Corp., Austin, Tex.) which allows for simultaneous measurement of multiple biomarkers in serum in order to accurately discriminate cancer status with only a moderate number of samples. To our knowledge, this is the largest panel of cytokine markers to be examined simultaneously in pancreatic cancer.

Identified below are serological markers comprising cytokine, growth and angiogenic factors useful in the detection of pancreatic cancer. The serological markers include IP-10, HGF, IL-6, IL-8, βFGF, IL-12p40, IFNγ, TNFα, TNFRI, TNFRII, Eotaxin, MCP-1, MIP-1α, MIP-1β, EGF and CA 19-9.

In one embodiment of the present invention, a method of diagnosing the presence of pancreatic cancer in a patient is provided. Eotaxin and MCP-1 are under-expressed in patients with pancreatic cancer, as compared to control individuals, whereas IP-10, HGF, IL-8, βFGF, IL-12p40, TNFRI, TNFRII, and CA 19-9 are over-expressed in those patients. As such, there is a very high likelihood that a patient exhibiting two or more, three or more, four or more, five or more, six or more, seven or more, eight or more, or nine or more of the following parameters: Eotaxin_{LO} and MCP-1_{LO}, IP-10_{HI}, HGF_{HI}, IL-8_{HI}, bFGF_{HI}, IL-12p40_{HI}, TNFRI_{HI}, TNFRII_{HI}, and CA 19-9_{HI}, compared to control individuals, has pancreatic cancer.

Additionally, a method to differentiate patients with pancreatic cancer and patients with chronic pancreatitis is provided. IL-6, IL-8, IFNγ, TNFα, Eotaxin, MCP-1, MIP-1α, MIP-1β, and EGF are under-expressed in patients with pancreatic cancer compared to patients with chronic pancreatitis, whereas IP-10 is over-expressed in patients with pancreatic cancer. Thus, there is a very high likelihood that a patient exhibiting two or more, three or more, four or more, five or more, six or more, seven or more, eight or more, or nine or more of the following parameters: IL-6_{LO}, IL-8_{LO}, IFNγ_{LO}, TNFα_{LO}, Eotaxin_{LO}, MCP-1_{LO}, MIP-1α_{LO}, MIP-1β_{LO}, EGF_{LO} and IP-10_{HI}, compared to patients with chronic pancreatitis, has pancreatic cancer.

In a further embodiment of the present invention, a method is provided comprised of predicting the onset of clinical pancreatic cancer, comprising determining the change in concentration at two or more time points of two or more, three or more, four or more, five or more, six or more, seven or more, eight or more, or nine or more of IP-10, HGF, IL-8, bFGF, IL-12p40, TNFRI, TNFRII, Eotaxin, MCP-1 and CA 19-9 in the patient's blood between the two time points, wherein an increase in the concentration of IP-10, HGF, IL-8, βFGF, IL-12p40, TNFRI, TNFRII, and CA 19-9, and a decrease in the concentration of Eotaxin and MCP-1 in the patient's blood between the two time points are predictive of the onset of pancreatic cancer.

In still a further embodiment of the present invention, a method for comparing the serum levels of the markers set forth herein in a blood marker panel of a patient with levels of the same markers in healthy matched controls or patients with chronic pancreatitis is provided comprised of applying statistical methods as set forth below.

Also provided is an array comprised of binding reagent types specific to any two or more, three or more, four or more, five or more, six or more, seven or more, eight or more, nine or more, ten or more, eleven or more, twelve or more, thirteen or more, fourteen or more, or fifteen or more of IP-10, HGF, IL-6, IL-8, βFGF, IL-12p40, IFNγ, TNFα, TNFRI, TNFRII, Eotaxin, MCP-1, MIP-1α, MIP-1β, EGF and CA 19-9, wherein each binding reagent type is attached independently to one or more discrete locations on one or more surfaces of one or more substrates. The substrates may be beads comprising an identifiable marker, wherein each binding reagent type is attached to a bead comprising a different identifiable marker than beads to which a different binding reagent is attached. The identifiable marker may comprise a fluorescent compound or a quantum dot.

To classify patients as either normal controls or pancreatic cancer cases, a variety of different classification methods can be implemented including logistic regression, classification trees, and neural networks. All analyses can be conducted using S-Plus statistical software. Each of the classification methods, which are described in further detail in the subsequent paragraphs, are implemented using 10-fold cross-validation (Efron and Tibshirani, 2000) to minimize bias of resulting classification rates. Classification accuracy is judged via the overall classification rate, sensitivity, specificity, and the receiver operating characteristic (ROC) curve. The ROC curve plots the sensitivity by 1-specificity across a range of cut-points. In other words, analysis begins by classifying all patients as a case and then the required predicted probability from 0.0 to 1.0 is increased (in 0.01 increments).

In each case, all estimates of classification accuracy (including the ROC curves) are calculated within the framework of 10-fold cross-validation. For each of the classification methods, the number of predictor variables is limited based on a univariate Wilcoxon rank-sum test, which assesses the significance of the difference in ranks between cases and controls for the given marker. The rank-sum test is the non-parametric analog to the two-sample unpaired t-test. In the case of classification trees (which automatically include a variable selection procedure as described in subsequent paragraphs), classification results are obtained using both the entire set of variables and those that are statistically significant with the Wilcoxon test.

Ten-fold cross-validation was implemented by first randomly partitioning the data into ten subsets. The same ten subsets were utilized for each of the subsequently described classification methods, so that classification results are comparable across different methods. The first nine subsets then are used to fit the model, and the last subset is used to calculate classification rates. The process is repeated ten times with a different subset selected each time for testing and the remaining subsets used for training.

Classification trees (Brieman, et al., 1984) first were used to predict cancer status. Classification trees are a non-parametric classification method that divide subjects into homogeneous subgroups of decreasing size and assign a probability of the given outcome to each group. More specifically, the method uses a technique called recursive partitioning, which searches the range of each potential predictor or marker, and finds the split which best divides the data into cases and controls. The process continues until the outcome is perfectly divided or the data are too sparse (e.g. n<5) for further classification. The proportion of cases in the final resulting subsets (i.e. terminal nodes) is used as the estimated predicted probability for corresponding test set observations. Results of the classification analysis also can be visually displayed using a decision tree to show the specific classification rules.

Logistic regression then is implemented to classify cases from controls. The logistic model is a standard parametric approach for classification of binary outcomes that calculates the predicted probability of an event (pancreatic cancer) as the logistic function of the weighted sum of the predictor variables, where the logistic function is defined as ƒ(z)=(1+e^{-z})⁻¹. For the logistic model, the set of predictor variables first is limited to those markers which are identified as statistically significant (p<0.05) from the rank-sum test.

Feed-forward neural networks also are implemented for classification analysis. Neural networks are an inherently non-linear parametric method that are universal approximators and may produce more accurate classification than standard methods such as logistic regression. The network response function can be stated as

$$\hat{y} = f ( {α}_{0} + {\sum }_{j} {α}_{j} f ( {β}_{0 j} + {\sum }_{i} {β}_{ij} {x}_{i} ) ) ,$$

where ƒ again is the logistic function and each

$$f ( {β}_{0 j} + {\sum }_{i} {β}_{ij} {x}_{i} )$$

is referred to as the j^{th} hidden unit. The model therefore is related to the logistic model, except that the logistic function of the weighted sum of separate logistic functions is taken. The model therefore is an inherently non-linear function of the data which implicitly fits interactions and non-linear terms (which can be formally shown via a Taylor's series expansion (Landsittel, et al., 2002).

In a typical study, the number of hidden units can be varied, for example, and without limitation, from a minimum of two to a maximum of 30 (where classification results appear to stabilize). A weight decay term (of 0.01), which is a penalized likelihood function, also can be incorporated to improve model fit and generalizability. The S-Plus algorithm uses an iterative fitting method based on maximizing the likelihood to calculate the optimal coefficients. The maximum number of iterations can be increased, for example, and without limitation, to 1,000 (from the default value of 100).

It is understood that these _{LO} and _{HI} values are approximate and are derived statistically. By using other statistical methods to detect the relative levels of each factor and to define the critical values for _{HI} and _{LO}, values slightly above or below, typically within one standard deviation of those approximate values might be considered as statistically significant values for distinguishing the _{LO} or _{HI} state from normal. For this reason, the word “about” is used in connection with the stated values. “Statistical classification methods” are used to identify markers capable of discriminating normal patients and patients with benign growths with ovarian cancer patients, and are used to determine critical blood values for each marker for discriminating such patients. Three particular statistical methods were used to identify discriminating markers and panels thereof. These statistical methods include: 1) linear regression; 2) classification tree methods (CART), along with CHAID and QUEST; and 3) statistical machine learning to optimize the unbiased performance of algorithms for predicting the masked class labels. Each of these statistical methods are well-known to those of ordinary skill in the field of biostatistics and can be performed as a process in a computer. A large number of software products are available commercially to implement statistical methods, such as, without limitation, S-PLUS®, commercially available from Insightful Corporation of Seattle, Wash.

By identifying markers present in pancreatic cancer patients and statistical methods useful in identifying which markers and groups of markers are useful in identifying pancreatic cancer patients, a person of ordinary skill in the art, based on the disclosure herein, can identify panels that provide superior selectivity and sensitivity. Examples of panels providing excellent discriminatory capability include, without limitation, IP-10, HGF, IL-6, IL-8, bFGF, IL-12p40, IFNγ, TNFα, TNFRI, TNFRII, Eotaxin, MCP-1, MIP-1α, MIP-1β, EGF and CA 19-9.

It will be recognized by those of ordinary skill in the field of biostatistics, that the number of markers in any given panel may be different depending on the combination of markers. With optimum sensitivity as specificity being the goal, one panel may include two markers, while another may include eight, both yielding similar results.

The term “binding reagent” and like terms, refers to any compound, composition or molecule capable of specifically or substantially specifically (that is with limited cross-reactivity) binding another compound or molecule, which, in the case of immune-recognition is an epitope. A “binding reagent type” is a binding reagent or population thereof having a single specificity. The binding reagents typically are antibodies, preferably monoclonal antibodies, or derivatives or analogs thereof, but also include, without limitation: Fv fragments; single chain Fv (scFv) fragments; Fab′ fragments; F(ab′)2 fragments; humanized antibodies and antibody fragments; camelized antibodies and antibody fragments; and multivalent versions of the foregoing. Multivalent binding reagents also may be used, as appropriate, including without limitation: monospecific or bispecific antibodies, such as disulfide stabilized Fv fragments, scFv tandems ((scFv)2 fragments), diabodies, tribodies or tetrabodies, which typically are covalently linked or otherwise stabilized (i.e., leucine zipper or helix stabilized) scFv fragments. “Binding reagents” also include aptamers, as are described in the art.

Methods of making antigen-specific binding reagents, including antibodies and their derivatives and analogs and aptamers, are well known in the art. Polyclonal antibodies can be generated by immunization of an animal. Monoclonal antibodies can be prepared according to standard (hybridoma) methodology. Antibody derivatives and analogs, including humanized antibodies can be prepared recombinantly by isolating a DNA fragment from DNA encoding a monoclonal antibody and subcloning the appropriate V regions into an appropriate expression vector according to standard methods. Phage display and aptamer technology is described in the literature and permit in vitro clonal amplification of antigen-specific binding reagents with very affinity low cross-reactivity. Phage display reagents and systems are available commercially, and include the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Fla. Aptamer technology is described for example and without limitation in U.S. Pat. Nos. 5,270,163, 5,475,096, 5,840,867 and 6,544,776.

The Luminex LabMAP bead-type immunoassay described below is an example of a sandwich assay. The term “sandwich assay” refers to an immunoassay where the antigen is sandwiched between two binding reagents, which typically are antibodies. The first binding reagent/antibody being attached to a surface and the second binding reagent/antibody comprising a detectable group. Examples of detectable groups include, without limitation, fluorochromes; enzymes; or epitopes for binding a second binding reagent, i.e., when the second binding reagent/antibody is a mouse antibody, which is detected by a fluorescently-labeled anti-mouse antibody, for example an antigen or a member of a binding pair, such as biotin. The surface may be a planar surface, such as in the case of a typical grid-type array, for example, without limitation, 96-well plates and planar microarrays, as described herein, or a non-planar surface, as with coated bead array technologies, where each “species” of bead is labeled with, for example, a fluorochrome, such as the Luminex technology described herein and in U.S. Pat. Nos. 6,599,331, 6,592,822 and 6,268,222, or quantum dot technology, for example, as described in U.S. Pat. No. 6,306,610.

The LabMAP system incorporates polystyrene microspheres that are dyed internally with two spectrally distinct fluorochromes. Using precise ratios of these fluorochromes, an array is created consisting of 100 different microsphere sets with specific spectral addresses. Each microsphere set can possess a different reactant on its surface. Because microsphere sets can be distinguished by their spectral addresses, they can be combined, allowing up to 100 different analytes to be measured simultaneously in a single reaction vessel. A third fluorochrome coupled to a reporter molecule quantifies the biomolecular interaction that has occurred at the microsphere surface. Microspheres are interrogated individually in a rapidly flowing fluid stream as they pass by two separate lasers in the Luminex analyzer. High-speed digital signal processing classifies the microsphere based on its spectral address and quantifies the reaction on the surface in a few seconds per sample.

For the assays described herein, the bead-type immunoassays are preferable for a number of reasons. As compared to ELISAs, costs and throughput are far superior. As compared to typical planar antibody microarray technology (for example, in the nature of the BD Clontech Antibody arrays, commercially available form BD Biosciences Clontech of Palo Alto, Calif.), the beads are far superior for quantification purposes because the bead technology does not require pre-processing or titering of the plasma or serum sample, with its inherent difficulties in reproducibility, cost and technician time. For this reason, although other immunoassays, such as ELISA, RIA and antibody microarray technologies, are capable of use in the context of the present invention, they are not preferred. As used herein, “immunoassays” refer to immune assays, typically, but not exclusively, sandwich assays, capable of detecting and quantifying desired blood markers simultaneously, namely IP-10, HGF, IL-6, IL-8, bFGF, IL-12p40, TNFRI, TNFRII, IFNγ, TNFα, Eotaxin, MCP-1, MIP-1α, MIP-1β, EGF and CA 19-9. Data generated from an assay to determine blood levels of these markers can be used to determine the likelihood of pancreatic cancer in the patient. As shown herein, if serum levels of markers in a blood marker panel from a patient of IP-10, HGF, IL-8, bFGF, IL-12p40, TNFRI, TNFRII, and CA 19-9 are significantly increased, and serum levels of Eotaxin and MCP-1 are significantly decreased, compared to healthy matched controls, then there is a very high likelihood that the patient has pancreatic cancer. Additionally, if serum levels of markers in a blood marker panel from a patient of IL-6, IL-8, IFNγ, TNFα, Eotaxin, MCP-1, MIP-1α, MIP-1β, and EGF are significantly decreased, and serum levels of IP-10 are significantly increased, compared to patients with chronic pancreatitis, then there is a very high likelihood that the patient has pancreatic cancer.

Data generated from an assay to determine blood levels of two, three or four or more of the markers IP-10, HGF, IL-6, IL-8, bFGF, IL-12p40, IFNγ, TNFα, TNFRI, TNFRII, Eotaxin, MCP-1, MIP-1α, MIP-1β, EGF and CA 19-9 can be used to determine the likelihood of pancreatic cancer in the patient. As shown herein, if any two or more, typically three or four of the following conditions are met in a patient's blood, Eotaxin_{LO} and MCP-1_{LO}, IP-10_{HI}, HGF_{HI}, IL-8_{HI}, bFGF_{HI}, IL-12p40_{HI}, TNFRI, TNFRII_{HI}, and CA 19-9_{HI}, compared to control individuals, there is a very high likelihood that the patient has pancreatic cancer. Further, as shown herein, if any two or more, typically three or four of the following conditions are met in a patient's blood, IL-6_{LO}, IL-8_{LO}, IFNγ_{LO}, TNFα_{LO}, Eotaxin_{LO}, MCP-1_{LO}, MIP-1α_{LO}, MIP-1β_{LO}, EGF_{LO} and IP-10_{HI}, compared to patients with chronic pancreatitis, there is a very high likelihood that the patient has pancreatic cancer. In one embodiment, if any three or more, preferably three or four of the following conditions are met in a patient's blood, Eotaxin_{LO} and MCP-1_{LO}, IP-10_{HI}, HGF_{HI}, IL-8_{HI}, βFGF_{HI}, IL-12p40_{HI, TNFRI}_{HI}, TNFRII_{HI}, and CA 19-9_{HI}, compared to control individuals, there also is a very high likelihood that the patient has pancreatic cancer; and if any three or more, preferably three or four of the following conditions are met in a patient's blood, IL-6_{LO}, IL-8_{LO}, IFNγ_{LO}, TNFα_{LO}, Eotaxin_{LO}, MCP-1_{LO}, MIP-1α_{LO}, MIP-1β_{LO}, EGF_{LO} and IP-10_{HI}, compared to patients with chronic pancreatitis, there also is a very high likelihood that the patient has pancreatic cancer.

In the context of the present disclosure, “blood” includes any blood fraction, for example serum, which can be analyzed according to the methods described herein. Serum is a standard blood fraction that can be tested, and is tested in the Examples below. By measuring blood levels of a particular marker, it is meant that any appropriate blood fraction can be tested to determine blood levels and that data can be reported as a value present in that fraction. As a non-limiting example, the blood levels of a marker can be presented as 50 pg/mL serum.

As described above, methods for diagnosing pancreatic cancer by determining levels of specific identified blood markers are provided. Also provided are methods of detecting preclinical pancreatic cancer, comprising determining the presence and/or velocity of specific identified markers in a patient's blood. By velocity, it is meant changes in the concentration of the marker in a patient's blood over time.

The methods of the present invention will be described in more detail in the following non-limiting example.

### EXAMPLE 1

### Multianalyte Profiling of Serum Cytokines for Detection of Pancreatic Cancer

1. Patient Population, Materials and Methods

Patient Populations. Serum samples from 54 patients diagnosed with pancreatic cancer, 22 patients with chronic pancreatitis, and 26 healthy age- and sex- and smoking status-matched controls were tested. Serum samples from patients with documented adenocarcinoma of the pancreas were collected under an IRB approved protocol. Breakdown of their disease stage was Stage 1=4, Stage IIA=7, Stage IIB=16, Stage III=12, Stage IV=15. Serum samples from patients with chronic pancreatitis were obtained from the University of Pittsburgh, Division of Gastroenterology under a separate IRB approved protocol. Healthy controls were recruited as a part of ongoing translational research studies within the UPCI Early Detection Research Network/Biomarker Detection Laboratory (EDRN/BDL). Written informed consent was obtained from each subject before sample collection. All samples from the three populations were drawn, processed, and stored under stringent conditions as described below.

Peripheral blood samples were collected following informed consent using standard venipuncture techniques into sterile 10 ml BD Vacutainer™ glass serum (red top) tubes (BD, Franklin Lakes, N.J.) and left to stand undisturbed for 30 minutes at room temperature. The tubes then were spun at room temperature at 20×100 rpm for 10 minutes in a Sorvall benchtop centrifuge. The serum fraction then was carefully collected by pipetting into a pre-chilled tube on ice and mixed to ensure homogeneity of the serum sample. The serum then was divided into 1.0 ml aliquots in pre-chilled 1.8 ml Cryovial tubes on ice. The aliquots then were stored at −80° C. or below. Processing time from phlebotomy to freezing at −80° C. was within one hour. Immediately prior to analysis, serum aliquots were thawed on ice with intermittent agitation to avoid the formation of precipitate. No more than two freeze-thaw cycles were allowed for each sample.

Development of LabMAP™ Assays. The LabMAP™ assay for CA 19-9 was developed in our laboratory essentially as described previously (Gorelik, E. et al., Multiplexed Immunobead-Based Cytokine Profiling for Early Detection of Ovarian Cancer, Cancer Epidemiology Biomarkers and Prevention, In Press, 2004). For each LabMAP™ assay, a proprietary combination of two specific antibodies, monoclonal capture and polyclonal detection, was utilized. The detection antibody was biotinylated using the EZ-Link Sulfo-NHS-Biotinylation Kit (Pierce, Rockford, Ill.) according to the manufacturer's protocol. The capture antibody was covalently coupled to individually spectrally addressed carboxylated polystyrene microspheres purchased from Luminex Corp. The minimum detection level for CA 19-9 was <3.3 pg/ml. Inter-assay variability, expressed as a coefficient of variation (CV), was calculated based on the average for ten patient samples and standards that were measured in four separate assays. The inter-assay variability within the replicates presented as an average CV was 8.7-11.2% (data not shown). Intra-assay variability was evaluated by testing quadruplicates of each standard and ten samples measured three times. The CVs of these samples were between 6.9 and 9.8% (data not shown). In addition, the percent recovery from serum was 96-98% and correlations with standard ELISAs (Calbiotech, Spring Valley, Calif.) were 92-94%.

Cytokine Multiplexed Assay. A 31-plex assay for IL-1b, IL-2, IL-4, IL-5, IL-6, IL-8, IL-10, IL-12p40, IL-13, IL-15, IL-17, IL-18, TNFα, IFNγ, IFNα, GM-CSF, G-CSF, MIP-1α, MIP-1β, MCP-1, Eotaxin, RANTES, EGF, VEGF, βFGF, HGF, IP-10, DR5, TNFRI, TNFRII, MIG-1 was performed on each serum sample using kits purchased from BioSource International (Camarillo, Calif.). The LabMAP™ serum assays were performed in 96-well microplate format as described above.

Statistical Analysis of Data. Descriptive statistics and graphical displays (i.e., dot plots) were prepared to show the distribution of the serum level of each marker for each disease state. The Wilcoxon rank-sum test was used to evaluate the significance of differences in marker expression between each disease state. Spearman's (nonparametric) rank correlation also was calculated to quantify the relationships between each pair of markers.

Discrimination of pancreatic cancer status was accomplished using classification trees (CART) (Brieman, F. J et al., Classification and Regression Trees, 1984, Monterey: Wadsworth and Brooks/Cole) implemented through S-Plus statistical software (Venables, W. et al., Modern applied statistics with S-plus, 1997, New York: Springer-Verlag), which classifies subjects into homogeneous subgroups of decreasing size and assigns a probability of the given outcome to each group. These groups then are drawn on a decision tree to show the specific rules used for classification. Comparisons were repeated for pancreatic cancer versus normal controls, and pancreatic cancer versus non-acute pancreatitis.

For comparisons of cancer versus normal controls, and cancer versus chronic pancreatitis, subjects with a predicted probability greater than or equal to 0.5 (using the classification tree model) were classified as cancerous, and all others (predicted probability less than 0.5) as non-cancerous (i.e., controls or chronic pancreatitis). To appropriately evaluate classification results, 10-fold cross-validation (Tibshirani, R. et al., Statist. Applic. Genet. Mol. Biol., 1 2002; Efron, R. et al., J. Amer. Statist. Associated. 96:1151-1160, 2001), also was implemented to provide a more unbiased measure of classification accuracy (as opposed to simply evaluating classification results on the same data used to fit the model, which is known to be optimistically biased and prone to overfitting). Sensitivity, specificity, and the overall classification rate were calculated to quantify classification accuracy. The classification trees presented for each comparison represent the model fit to the entire data set. The ROC curves utilized 10-fold cross-validation to produce all classification results.

2. Results

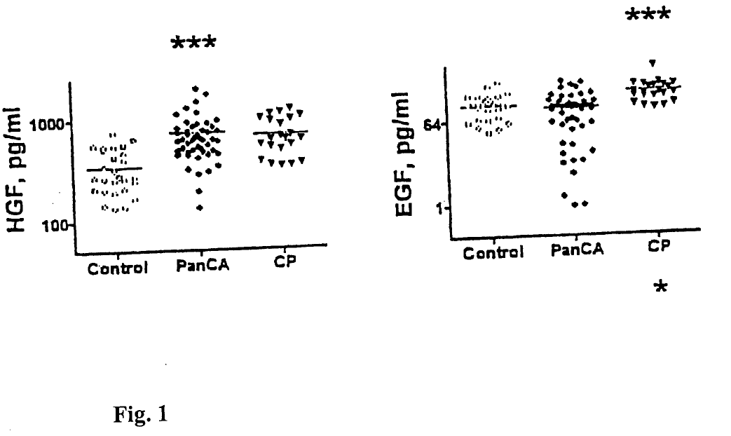

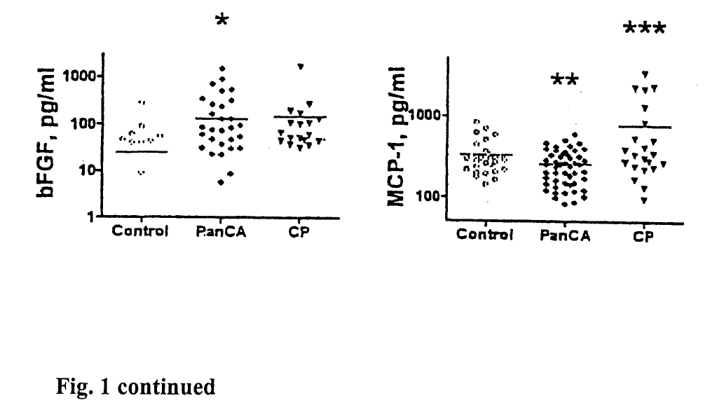

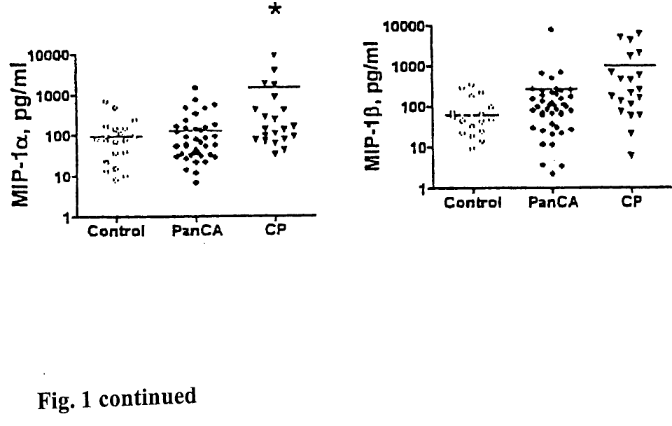

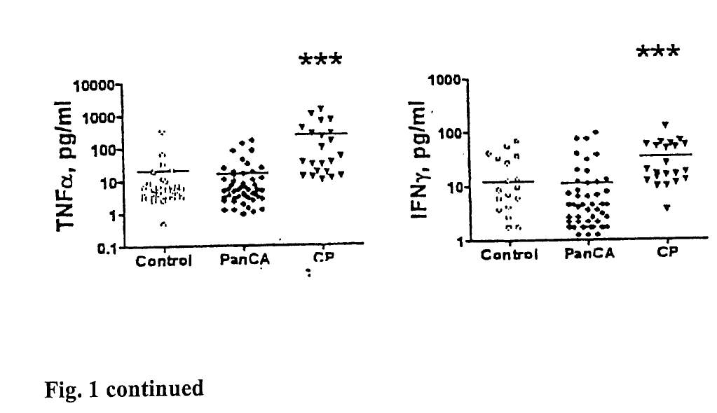

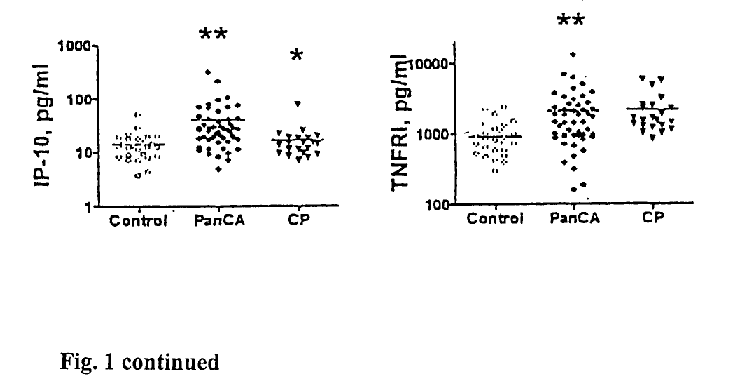

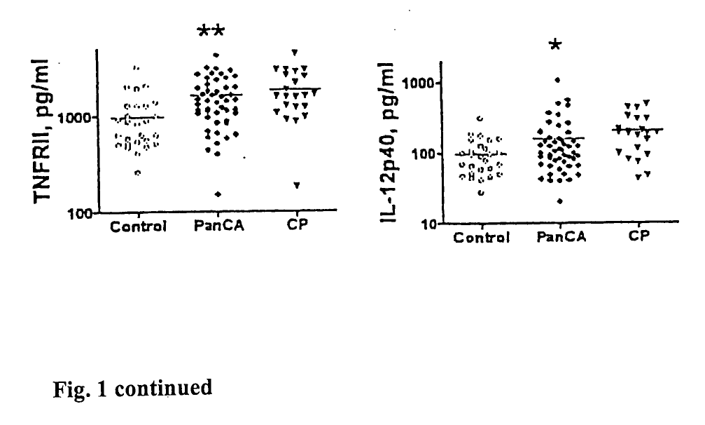

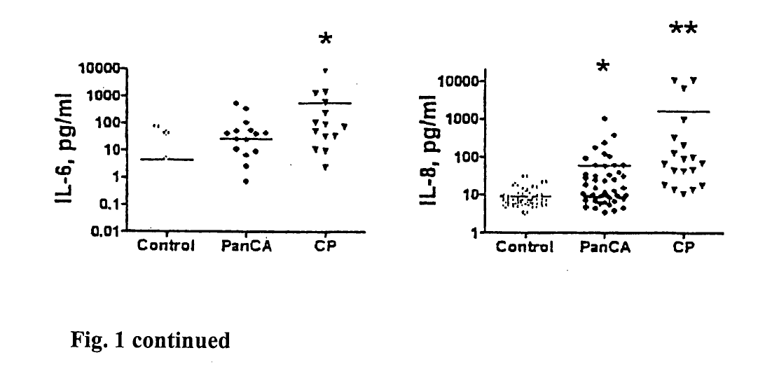

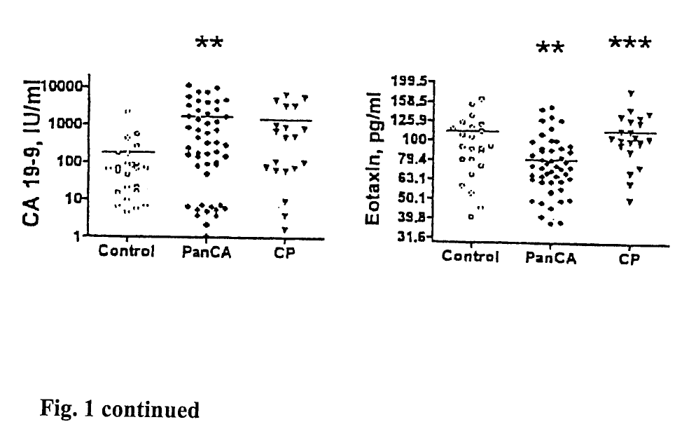

LabMAP™-Based Analysis of Serum Concentrations of Cytokines and Cancer Markers in Pancreatic Cancer Patients. Concentrations of 31 different serum markers belonging to different biological functional groups, and CA 19-9 were evaluated in a multiplexed assay using LabMAP™ technology, in serum samples of patients from three clinical groups: pancreatic cancer patients, patients with chronic pancreatitis, and control healthy subjects who were matched to disease groups by age, sex and smoking status. The results of the multiplex analysis are presented in Table 1 and FIG. 1, which show the summary statistics, including the mean, standard error, median, and range, for each marker.

Pancreatic Cancer vs. Controls. Multiplexed assay of 31 serum cytokines revealed a group of nine cytokines whose concentrations were significantly different in patients with pancreatic cancer as compared to healthy controls. Specifically, serum concentrations of IP-10, HGF, IL-8, FGF, IL-12p40, TNFRI and TNFRII were found to be significantly higher in pancreatic cancer patients as compared to controls (P<0.05 -P<0.001) (Table 1, FIG. 1). Concentration of MCP-1 and Eotaxin were significantly (P<0.001) lower in pancreatic cancer patients as compared to controls (Table 1, FIG. 1). In addition, as expected, serum concentrations of CA 19-9 were found to be significantly higher in pancreatic cancer patients as compared to controls (P<0.05 -P<0.001). These candidate biomarkers were selected for further statistical analysis.

Pancreatic Cancer vs. Chronic Pancreatitis. Serum cytokine concentrations in patients with pancreatic cancer were measured and compared to those in patients with chronic pancreatitis. This comparison identified 11 markers demonstrating significant differences in serum concentrations between these two clinical groups. Serum concentration of IP-10 was found to be significantly higher in pancreatic cancer patients as compared to chronic pancreatitis patients (P<0.05) (Table 1, FIG. 1). Concentrations of IL-6, IL-8, IFNγ, TNFα, Eotaxin, MCP-1, MIP-1α, MIP-1β, IP-10, and EGF were significantly lower (P<0.05-P<0.001) in pancreatic cancer patients as compared to patients with chronic pancreatitis (Table 1, FIG. 1). Concentrations of CA 19-9 were not significantly different between these two groups. Biomarkers that showed a statistical significance between groups with pancreatic cancer and chronic pancreatitis were selected for further statistical analysis.

Correlation Between Biomarkers. Analysis of correlations between individual cytokine markers that are associated with pancreatic cancer using Spearman rank correlation method revealed that IP-10, HGF, IL-8, βFGF, MCP-1, and CA 19-9 were relatively uncorrelated, i.e. correlation coefficients were below 0.5 (data not shown). Of the remaining markers, Eotaxin correlated with IL-12p40 (r=0.5), and TNFRI correlated with TNFRII (r=0.68).

Statistical Analysis of Serum Cytokines as Pancreatic Cancer Biomarkers Comparison of Controls versus Pancreatic Cancer Cases. LabMAP™ analysis identified ten markers demonstrating significant differences between pancreatic cancer patients and healthy controls. These markers were used singly for classification analysis to distinguish pancreatic cancer from controls. Results show that the individual markers led to only moderately accurate prediction of pancreatic cancer. Only IP-10, Eotaxin, IL-12p40 and IL-8, when considered individually, correctly classified over 80% of the test set subjects (Table 2).

Next, CART methodology was used for discriminating controls from pancreatic cancer. All these markers were entered as potential variables in the classification tree algorithm. The resulting classification tree selected by S-Plus software included HGF, MCP-1, IP-10 and Eotaxin. Interestingly, the S-Plus program did not include CA 19-9 in the classification tree. Classification rates then were obtained for the given set of markers (again based on classification tree models and 10-fold cross-validation). The overall classification rate for discriminating pancreatic cancer cases from controls was 88% ( 66/75), with a sensitivity of 86% ( 42/49) and a specificity of 92% ( 24/26). FIG. 2A represents the ROC curve (which again uses 10-fold cross-validation to calculate predicted values). The data revealed a relatively high specificity across a range of high sensitivities. Several other marker combinations offered similar classification results, i.e., HGF, MCP-1, IFNγ, TNFRII, and Eotaxin, or HGF, MCP-1, IP-10, TNFα, and EGF, etc.

The classification analysis then was repeated using only CA 19-9 (in a classification tree model with 10-fold cross-validation) to predict cancer status. The overall classification rate for discriminating pancreatic cancer cases from controls was 77% ( 58/75), with a sensitivity of 88% ( 43/49) and a specificity of 58% ( 15/26). The ROC curve (FIG. 2A), which again uses 10-fold cross-validation to calculate predicted values, showed relatively high specificity for sensitivities at or below 80%, but showed a substantial drop when the sensitivity was increased above 80%.

Comparison of Chronic Pancreatitis versus Pancreatic Cancer Cases. All markers were entered as potential variables in the classification tree algorithm. This analysis resulted in the model that includes IFNγ, TNFα, IL-8, IP-10 and TNFRII. Using the previously described classification tree and the 10-fold cross-validation approach, the data then were classified as either chronic pancreatitis or pancreatic cancer. Results showed very accurate classification; 48 out of 49 pancreatic cancer cases were correctly predicted to be pancreatic cancer. Nineteen of 22 chronic pancreatitis subjects were correctly classified as chronic pancreatitis. This equated to 98% sensitivity and 86% specificity. Overall, 94% of the subjects were correctly classified. FIG. 2B represents the ROC curve that shows a high specificity across any reasonable range of sensitivities.

The classification analysis again was repeated using only CA 19-9 (in a classification tree model with 10-fold cross-validation) to predict cancer status. The overall classification rate for classifying pancreatic cancer cases from chronic pancreatitis was 77% ( 58/75), with a sensitivity of 94% ( 46/49) and a specificity of 41% ( 9/22). The ROC for CA 19-9 (FIG. 3B) showed relatively high specificity for sensitivities at near 80%, but showed a substantial drop when the sensitivity was increased above 80%.

3. Discussion

Multiplexed LabMAP™ technology was utilized for analysis of 31 cytokines and CA 19-9 in sera of patients with pancreatic cancer in comparison with patients with chronic pancreatitis and matched healthy controls. To our knowledge, this is the largest panel of cytokine markers to be examined simultaneously in pancreatic cancer. The sensitivity of the LabMAP™ assays were comparable to ELISA and RIA [R. T. Carson, R. T. et al., Immunol. Methods, 227:41-52, 1999). Circulating levels of all 31 proteins in healthy patients were very similar to those measured by ELISA or RIA and reported in previously published observations (Penson, R. T. et al., Int. J. Gynecol. Cancer, 10:33-41, 2000).

Nine circulating proteins were identified that showed an association with pancreatic cancer versus healthy matched controls: IP-10, IL-8, HGF, βFGF, IL-12p40, TNFRI, Eotaxin, MCP-1, and CA 19-9. Two patterns of changes were observed: the serum concentrations of IL-8, βFGF, HGF, IP-10, IL-12p40, TNFRI, TNFRII, and CA 19-9, were higher; whereas concentrations of Eotaxin and MCP-1 were decreased in patients with pancreatic cancer in comparison to the controls. Observations of elevated serum levels of IP-10, IL-8, IL-12p40, and TNFRI support the concept that pancreatic cancer has a strong inflammatory component and help refine our understanding of the magnitude and scope of these inflammatory changes. However, Eotaxin and MCP-1, which normally are elevated during inflammation were decreased in pancreatic cancer. This may be due to active consumption of these cytokines by immune or tumor cells. Interestingly, in chronic pancreatitis, mean circulating Eotaxin concentrations did not differ from controls, and serum MCP-1 concentrations were significantly higher than in the controls, indicating that lower Eotaxin and MCP-1 levels were specific for pancreatic neoplasia, and not just for pancreatic abnormality.

LabMAP™ technology also was used to examine serum cytokine profiles in patients with six other cancers: ovarian, breast, lung, esophageal, hepatocellular (HCC) and melanoma (Gorelik, E. et al., Multiplexed Immunobead-Based Cytokine Profiling for Early Detection of Ovarian Cancer, Cancer Epidemiology Biomarkers and Prevention, In Press, 2004, and inventors' unpublished observations). It appears that the serum cytokine profile of each of these cancers was unique. The only other cancer demonstrating increased serum IP-10 concentrations was HCC. To the best of the knowledge of the inventors, there are no published data on elevated serum concentrations of IP-10 in other cancers. Therefore, IP-10 may represent a cytokine that is relatively specific for pancreatic cancer. IP-10 may serve as a more reliable marker of gastrointestinal diseases than CA 19-9, because the latter also is expressed in gynecologic malignancies (Gadducci, A. et al., Eur. J. Gynaecol. Oncol., 11:127-133, 1990). In addition to pancreatic cancer, elevated serum concentrations of IL-12p40 were observed in melanoma and HCC (inventors' unpublished observations). Elevated concentrations of serum HGF are typical for gastrointestinal cancers, i.e. HCC, gastric and colon cancers (Yamagamim, H. et al., Cancer, 95: 824-34, 2002; Beppu, K. et al., Anticancer Res., 20: 1263-7, 2000; Fukuura, T. et al., Br. J. Cancer, 78: 454-9, 1998, and inventors' unpublished observations), as well as in inflammatory gastrointestinal and pancreatic diseases (Matsuno, M. et al., Res. Commun. Mol. Pathological. Pharmacol., 97: 25-37, 1997). In addition, elevated serum levels of HGF have been observed in prostate and small cell lung cancer (Naughton, M. et al., J. Urol., 165: 1325-8, 2001; Bharti, A. et al., Anticancer Res., 24: 1031-8, 2004), and in melanoma (inventor's unpublished observations). However, the inventors have not observed increased concentrations of serum HGF in ovarian or breast cancers, where CA 19-9 was significantly elevated. βFGF has been shown to be elevated in sera of patients with several cancers including colorectal, breast, ovarian, and renal carcinomas (Dirix, L. Y. et al., Br. J. Cancer, 76: 238-43, 1997) and HCC (inventors' unpublished observations). Serum TNFRI has been shown to be elevated in breast cancer and melanoma (Tesarova, P. et al., Med. Sci. Monit., 6: 661-7, 2000, and inventors' unpublished observations). Eotaxin and MCP-1 have been shown to be lower in several cancers, i.e. gastric cancer (Tonouchi, H. et al., Scand. J. Gastroenterol., 37: 830-3, 2002), as well as ovarian, breast and lung cancers (inventors' unpublished observations). IL-8 is the most non-specific cancer marker as it is elevated in most human cancers (Xie, K., Cytokine Growth Factor Rev., 12: 375-91, 2001), and inventors' unpublished observations). Therefore, each marker considered separately may be elevated in several cancers. However, multiplexed LabMAP™ technology allowed identification of combinations of these cytokines that appear to be unique for each particular cancer, and thus represents cancer “cytokine signatures.”

Statistical analysis demonstrated that although correlation of each of the identified markers with pancreatic cancer was modest when evaluated alone, a combined biomarker panel showed very strong association with malignant disease. Combinations of several serum markers as measured by LabMAP™ technology provided a sensitivity of 86% at a specificity of 92% for comparison of pancreatic cancer with healthy controls. As a diagnostic panel, these markers performed better than CA 19-9 alone in distinguishing pancreatic cancer from normal controls and chronic pancreatitis. Moreover, this panel has demonstrated higher performance than any published single pancreatic cancer-associated marker (Hayakawa, T. et al., Int. J. Pancreatol., 25: 23-9, 1999; Carpelan-Holmstrom, M. et al., Anticancer Res., 22: 2311-6, 2002), or marker combination, i.e. the combination of CA 19-9 with CEA and CA 72-4 marker (Hayakawa, T. et al., Int. J. Pancreatol., 25: 23-9, 1999; Carpelan-Holmstrom, M. et al., Anticancer Res., 22: 2311-6, 2002).

The ability to discriminate between patients with benign inflammatory conditions of the pancreas and malignancy is of significant clinical importance. Current diagnostic modalities are inadequate and result in approximately 10% of patients undergoing resection for suspected pancreatic cancer with benign final pathology. Analysis of serum biomarkers in patients with chronic pancreatitis versus pancreatic cancer patients demonstrated a significant increase in inflammatory cytokines, IL-6, IL-8, IFNγ, TNFα, Eotaxin, MCP-1, MIP-1α, MIP-1β, and EGF. In contrast, IP-10 concentrations were significantly higher in pancreatic cancer as compared with chronic pancreatitis. Combinations of several serum biomarkers as measured by LabMAP™ technology provided a sensitivity of 98% at a specificity of 96% for discrimination of pancreatic cancer from chronic pancreatitis. Thus, the multicytokine panel can serve as a very efficient discriminator between chronic pancreatitis and pancreatic cancer.

It is of interest to note that, when generating the classification tree for discrimination of pancreatic cancer from healthy controls, S-Plus software did not include CA 19-9. Furthermore, the software selected MCP-1 whose association with pancreatic cancer is relatively low as compared with other markers. Markers with the highest individual classification results typically are included in the overall model, but this is not necessarily always the case. First, the individual classification uses 10-fold cross-validation, and thus has a random component to achieving results. The “best” marker and the “next-best,” for instance, may actually be equal or in reverse order due to chance. Although using a 10-fold approach minimizes this possibility, it still may occur. Also, once the tree splits once, the markers are judged strictly on their discrimination within the resulting subsets, not over the entire data set.

For an estimate of the optimal classification tree, presented herein was a model fit to the entire data set, referred to as the overall model. It should be noted that the cross-validation procedure utilized herein produced a potentially different model for each of the ten randomly selected training data sets. Each of these ten classification trees, however, was either the same as, or subsets of likely similar to, the overall model. None of the ten models fit through the cross-validation procedure included any markers that were not in the overall model. Although some bias may result from this cross-validation procedure, as opposed to separate training and test sets, the latter approach typically is highly variable unless one has large sample sizes. With the given sample sizes available in this study, separate training and test sets would lead to more unstable estimates of sensitivity and specificity, because each observation can only be used for training or prediction. For the given data, the 10-fold cross-validation approach represents a reasonable alternative to at least partially avoid classification bias (imposed when the same data are used from both training and prediction), and estimate classification measures (e.g. sensitivity and specificity) with improved precision. This type of analysis demonstrated the ability to accurately discriminate cancer status with only a moderate number of samples.

It should be understood that the embodiments described herein are for illustrative purposes only and that various modifications or changes in light thereof will be suggested to persons skilled in the art and are to be included within the spirit and purview of this application.

TABLE 1
Summary statistics for serum cytokines by disease status

| | | | | | Min- | |
|---|---|---|---|---|---|---|
| Marker | Status | Mean | SE | Median | imum | Maximum |
| IL-6 | Chronic | 591.6 | 411.00 | 10.0 | 0.0 | 9022.4 |
| | Pancreatitis | | | | | |
| | Pancreatic | 26.9 | 13.56 | 0.0 | 0.0 | 560.0 |
| | Cancer | | | | | |
| | Controls | 4.3 | 3.08 | 0.0 | 0.0 | 72.0 |
| IL-8 | Chronic | 2872.6 | 985.18 | 93.0 | 10.6 | 11000.0 |
| | Pancreatitis | | | | | |
| | Pancreatic | 58.1 | 22.27 | 10.8 | 3.5 | 1026.0 |
| | Cancer | | | | | |
| | Controls | 9.0 | 1.09 | 6.9 | 3.3 | 28.8 |
| IFNγ | Chronic | 34.1 | 6.16 | 18.2 | 3.6 | 123.0 |
| | Pancreatitis | | | | | |
| | Pancreatic | 11.1 | 2.87 | 3.6 | 0.0 | 94.4 |
| | Cancer | | | | | |
| | Controls | 12.1 | 3.57 | 3.8 | 0.0 | 66.1 |
| TNFα | Chronic | 240.2 | 78.04 | 42.7 | 9.9 | 1364.2 |
| | Pancreatitis | | | | | |
| | Pancreatic | 16.2 | 4.61 | 4.8 | 0.0 | 162.5 |
| | Cancer | | | | | |
| | Controls | 20.4 | 12.12 | 5.2 | 0.4 | 317.7 |
| Eotaxin | Chronic | 112.8 | 8.44 | 103.3 | 49.8 | 240.9 |
| | Pancreatitis | | | | | |
| | Pancreatic | 80.2 | 4.80 | 73.1 | 9.1 | 208.8 |
| | Cancer | | | | | |
| | Controls | 111.5 | 10.98 | 98.1 | 40.2 | 274.7 |
| MCP-1 | Chronic | 773.8 | 195.03 | 341.3 | 94.4 | 3403.3 |
| | Pancreatitis | | | | | |
| | Pancreatic | 257.4 | 18.21 | 242.7 | 82.6 | 610.4 |
| | Cancer | | | | | |
| | Controls | 331.6 | 34.48 | 281.5 | 140.8 | 823.7 |
| MIP1α | Chronic | 1443.7 | 674.47 | 143.4 | 32.1 | 12420.5 |
| | Pancreatitis | | | | | |
| | Pancreatic | 127.2 | 35.61 | 36.5 | 0.0 | 1457.5 |
| | Cancer | | | | | |
| | Controls | 92.4 | 30.04 | 36.1 | 0.0 | 664.4 |
| MIP1β | Chronic | 2973.4 | 1774.2 | 192.3 | 0.0 | 35810.0 |
| | Pancreatitis | | | | | |
| | Pancreatic | 272.5 | 157.17 | 76.8 | 0.0 | 7744.3 |
| | Cancer | | | | | |
| | Controls | 60.7 | 17.58 | 27.3 | 0.0 | 332.4 |
| EGF | Chronic | 257.2 | 33.40 | 221.7 | 106.8 | 851.1 |
| | Pancreatitis | | | | | |
| | Pancreatic | 118.6 | 15.75 | 109.4 | 0.0 | 444.9 |
| | Cancer | | | | | |
| | Controls | 137.5 | 18.22 | 129.3 | 0.0 | 403.7 |
| bFGF | Chronic | 148.6 | 75.06 | 52.3 | 0.0 | 1693.6 |
| | Pancreatitis | | | | | |
| | Pancreatic | 131.2 | 39.59 | 22.9 | 0.0 | 1497.6 |
| | Cancer | | | | | |
| | Controls | 43.0 | 21.93 | 0.0 | 0.0 | 521.0 |
| HGF | Chronic | 683.2 | 59.65 | 606.5 | 330.1 | 1190.7 |
| | Pancreatitis | | | | | |
| | Pancreatic | 729.1 | 64.95 | 621.4 | 133.6 | 2646.5 |
| | Cancer | | | | | |
| | Controls | 338.6 | 33.57 | 265.9 | 133.9 | 728.8 |
| IL-12 | Chronic | 205.5 | 27.01 | 189.3 | 42.9 | 484.3 |
| p40 | Pancreatitis | | | | | |
| | Pancreatic | 161.0 | 25.34 | 99.9 | 20.5 | 1048.9 |
| | Cancer | | | | | |
| | Controls | 97.0 | 11.90 | 82.5 | 27.1 | 300.9 |
| TNFRI | Chronic | 2160.2 | 309.2 | 1567.7 | 845.3 | 5825.4 |
| | Pancreatitis | | | | | |
| | Pancreatic | 2085.3 | 315.6 | 1431.9 | 30.1 | 13182.9 |
| | Cancer | | | | | |
| | Controls | 909.7 | 108.4 | 769.7 | 94.6 | 2278.6 |
| TNFRII | Chronic | 1843.7 | 205.3 | 1576.9 | 180.5 | 4338.6 |
| | Pancreatitis | | | | | |
| | Pancreatic | 1621.8 | 143.0 | 1422.9 | 151.0 | 5129.6 |
| | Cancer | | | | | |
| | Controls | 966.4 | 129.4 | 714.4 | 255.3 | 3119.8 |
| IP-10 | Chronic | 16.9 | 3.019 | 14.5 | 7.0 | 76.4 |
| | Pancreatitis | | | | | |
| | Pancreatic | 40.7 | 7.53 | 25.3 | 4.8 | 315.9 |
| | Cancer | | | | | |
| | Controls | 14.2 | 1.78 | 12.3 | 3.7 | 48.7 |
| CA19-9 | Chronic | 1427.0 | 468.7 | 482.7 | 1.7 | 6623.0 |
| | Pancreatitis | | | | | |
| | Pancreatic | 1670.0 | 381.1 | 311.4 | 1.1 | 11231.0 |
| | Cancer | | | | | |
| | Controls | 177.5 | 85.4 | 62.2 | 4.4 | 2046.0 |

TABLE 2
Predictive values for individual serum
markers for pancreatic cancer

| | | | Correctly |
|---|---|---|---|
| Cytokine | Sensitivity | Specificity | Classified |
| IP-10 | 85.7% | 76.9% | 82.7% |
| Eotaxin | 93.9% | 57.7% | 81.3% |
| IL-8 | 89.8% | 65.4% | 81.3% |
| IL-12p40 | 61.5% | 91.8% | 81.3% |
| HGF | 87.8% | 65.4% | 80.0% |
| TNFRI | 81.6% | 76.9% | 80.0% |
| TNFRII | 81.2% | 75.0% | 78.6% |
| CA 19-9 | 87.8% | 57.7% | 77.3% |
| bFGF | 34.6% | 93.9% | 73.3% |
| MCP-1 | 79.6% | 57.7% | 72.0% |

1. A method of determining the presence of pancreatic cancer in a patient, comprising:
determining levels of markers in a blood marker panel, comprising two or more of IP-10, HGF, IL-8, βFGF, IL-12p40, TNFRI, TNFRII, Eotaxin, MCP-1 and CA 19-9 in a sample of the patient's blood, wherein the presence of two or more of the following conditions indicates the presence of pancreatic cancer in the patient: Eotaxin_{LO} and MCP-1_{LO}, IP-10_{HI}, HGF_{HI}, IL-8_{HI}, βFGF_{HI}, IL-12p40_{HI}, TNFRI_{HI}, TNFRII_{HI}, and CA 19-9_{HI}, compared to control individuals.

2. The method of claim 1, wherein the panel comprises 3 to 5 of IP-10, HGF, IL-8, βFGF, IL-12p40, TNFRI, TNFRII, Eotaxin, MCP-1 and CA 19-9.

3. The method of claim 1, wherein the panel comprises 4 of IP-10, HGF, IL-8, βFGF, IL-12p40, TNFRI, TNFRII, Eotaxin, MCP-1 and CA 19-9.

4. The method of claim 1, wherein the panel comprises 5 of IP-10, HGF, IL-8, βFGF, IL-12p40, TNFRI, TNFRII, Eotaxin, MCP-1 and CA 19-9.

5. The method of claim 1, wherein a multianalyte LabMap profiling technology is utilized that allows for simultaneous determination of the levels of markers in the blood marker panel.

6. The method of claim 1, further comprising comparing the levels of the two or more markers in the patient's blood with levels of the same markers in a control sample by applying a statistical method selected from the group consisting of linear regression analysis, classification tree analysis and heuristic naive Bayes analysis.

7. The method of claim 6, wherein the statistical method is performed by a computer process.

8. The method of claim 6, wherein the statistical method is a classification tree analysis.

9. The method of claim 6, wherein the blood marker panel generates a sensitivity of at least about 85% and a specificity of at least about 92% using the statistical method.

10. A method of differentiating patients with pancreatic cancer from patients with chronic pancreatitis, comprising:
determining levels of markers in a blood marker panel comprising two or more of IP-10, IL-6, IL-8, IFNγ, TNFα, Eotaxin, MCP-1, MIP-1α, MIP-1β, and EGF in a sample of the test patient's blood, wherein the presence of two or more of the following conditions indicates the presence of pancreatic cancer in the test patient: IL-6_{LO}, IL-8_{LO}, IFNγ_{LO}, TNFα_{LO}, Eotaxin_{LO}, MCP-1_{LO}, MIP-1α_{LO}, MIP-1β_{LO}, EGF_{LO} and IP-10_{HI}, compared to patients with chronic pancreatitis.

11. The method of claim 10, wherein the panel comprises 3 to 5 of IP-10, IL-6, IL-8, IFNγ, TNFα, Eotaxin, MCP-1, MIP-1α, MIP-1β, and EGF.

12. The method of claim 10, wherein the panel comprises 4 of IP-10, IL-6, IL-8, IFNγ, TNFα, Eotaxin, MCP-1, MIP-1α, MIP-1β, and EGF.

13. The method of claim 1, wherein the panel comprises 5 of IP-10, IL-6, IL-8, IFNγ, TNFα, Eotaxin, MCP-1, MIP-1α, MIP-1β, and EGF.

14. An array comprising binding reagent types specific to any two or more of IP-10, HGF, IL-6, IL-8, βFGF, IL-12p40, IFNγ, TNFα, TNFRI, TNFRII, Eotaxin, MCP-1, MIP-1α, MIP-1β, EGF and CA 19-9, wherein each binding reagent type is attached independently to one or more discrete locations on one or more surfaces of one or more substrates.

15. The array of claim 14, wherein the substrates are beads comprising an identifiable marker, wherein each binding reagent type is attached to a bead comprising a different identifiable marker than beads to which a different binding reagent is attached.

16. The array of claim 15, wherein the identifiable marker comprises a fluorescent compound.

17. The array of claim 15, wherein the identifiable marker comprises a quantum dot.

18. A method of predicting onset of clinical pancreatic cancer in a patient, comprising determining the change in serum levels at two or more time points of two or more of IP-10, HGF, IL-6, IL-8, βFGF, IL-12p40, IFNγ, TNFα, TNFRI, TNFRII, Eotaxin, MCP-1, MIP-1α, MIP-1β, EGF and CA 19-9 in the patient's blood, wherein an increase in the serum levels of IP-10, HGF, IL-8, βFGF, IL-12p40, TNFRI, TNFRII, and CA 19-9 in the patent's blood between the two time points and a decrease in the serum levels of Eotaxin and MCP-1 in the patient's blood between the two time points are predictive of the onset of pancreatic cancer.
